(19) United States
(12) Patent Application Publication
(10) Pub. No.: US 2014/0128461 A1
(43) Pub. Date: May 8, 2014

(54) USE OF A FOOD COMPOSITION IN THE TREATMENT AND/OR PREVENTION OF NEUROPATHIC PAIN INDUCED BY AN ANTICANCER AGENT

(71) Applicants: David Balayssac, Clermont-Ferrand (FR); Mathilde Bayet-Robert, Savasse (FR); Jérémy Ferrier, Aubiere (FR); Jacques Moulinoux, Rennes (FR)

(72) Inventors: David Balayssac, Clermont-Ferrand (FR); Mathilde Bayet-Robert, Savasse (FR); Jérémy Ferrier, Aubiere (FR); Jacques Moulinoux, Rennes (FR)

(21) Appl. No.: 13/990,702

(22) PCT Filed: Nov. 30, 2011
(86) PCT No.: PCT/EP2011/071439

§ 371 Date: Jan. 17, 2014

(30) Foreign Application Priority Data
Nov. 30, 2010 (FR) 1059932

(51) Int. Cl. A23L 1/29; A61K 31/282

(52) U.S. Cl. CPC A23L 1/296; A61K 31/282
USPC 514/492; 556/137; 426/648; 426/2

(57) ABSTRACT

The invention relates to the use of a low-polyamine-content food composition for use in the treatment and/or prevention of neuropathic pain induced by a platinum salt.

The invention concerns the use of a food composition in the treatment and/or prevention of neuropathic pain induced by an anticancer agent, in particular platinum salts and more particularly oxaliplatin. The invention also concerns particular treatment procedures with these anticancer agents for patients suffering from cancer.

Oxaliplatin (Eloxatin®) is an anticancer agent belonging to the family of platinum salts. At the current time, oxaliplatin is used in adjuvant chemotherapy for the treatment of colon cancers after resection, oesophageal carcinomas, gastric and pancreatic adenocarcinomas (Louvet et al. 2002), in some hepatocarcinomas (Yen et al. 2008) and some ovarian carcinomas (Chollet et al. 1996). Oxaliplatin has also exhibited therapeutic efficacy in non-small-cell lung cancers (Monnet et al. 1998) and non-Hodgkin lymphomas (Germann et al. 1999). At therapeutic doses, oxaliplatin leads to dose-limiting neurotoxicity that is very frequent. The neurotoxicity induced by oxaliplatin is displayed in two forms: acute and chronic.

The onset of acute neurotoxicity occurs during or just after an infusion of oxaliplatin in more than 90% of treated patients. It is characterized by paraesthesia and dysaesthesia of the limbs, of the perioral region, at times accompanied by motor symptoms such as cramps, tetanic cramps or myotonia (Pasetto et al. 2006; Kiernan 2007). These symptoms are all triggered or aggravated by exposure to cold. Most often they are reversible but reoccur at the time of further oxaliplatin infusion. The symptoms of thermal hyperalgesia (increased sensitivity to pain stimulus) on exposure to heat or cold are characteristic of neuropathy induced by oxaliplatin (Attal et al. 2009). Acute symptoms have been attributed to channelopathy, a functional disorder of membrane ion channels (Argyriou et al. 2008).

Accumulative chronic neurotoxicity follows after acute neurotoxicity. Chronic neurotoxicity develops progressively in 10 to 20% of patients after the administration of an accumulated oxaliplatin dose of 750 to 850 mg/m² and may require withdrawal of treatment (de Gramont et al. 2000; Andre et al. 2004). Sensory peripheral neuropathy is characterized by a persistent symptomatology between oxaliplatin treatment periods comprising extended paraesthesia, loss of superficial and deep sensitivity, ataxia and the onset of spontaneous pain.

At the current time there is no specific treatment for iatrogenic neuropathy. Neuropathic pain is often described as difficult to treat and resistant to conventional analgesic treatment. The treatment of these painful syndromes is chiefly based on the use of antidepressants and anticonvulsants which only provide partial relief of nociceptive disorders (Saif en al. 2005). Neuropathy induced by platinum salts does not only affect patients' quality of life since pauses in treatment, reductions in dose or treatment withdrawal may reduce chances of survival (Oldenburg et al. 2009). Within this setting, there is a real need for therapeutic innovation in the management of neuropathic pain induced by platinum salts.

Within this context the N-methyl-D-aspartate (NMDA) receptors are ionotropic glutamate receptors expressed on the surface of nerve cells along the nociceptive pathways. The NMDA receptors located the spinal cord play a role in the onset of neuropathic pain by initiating a physiopathological process called central sensitization (Woolf et al. 1991). Central sensitization leads to a state of post-synaptic hyperexcitability which may cause hyperalgesia and allodynia (Latremoliere et al. 2009). Preclinical trials in different animal models of traumatic and metabolic neuropathy have shown that the administration of NMDA receptor antagonists (such as MK801) can overcome painful hypersensitivity. However, the clinical use of these antagonists remains limited through the occurrence of major side effects of cognitive type such as partial amnesia, psychotomimetic effects and ataxia. To prevent neuropathic pain, it therefore appears necessary to adopt a therapeutic strategy based on modulating the pathological activation of the NMDA receptors without inhibiting their physiological activation.

Polyamines (spermine, spermidine, putrescine and cadaverine) are positively charged molecules which are essentially food-derived. Polyamines are involved in the painful process by positively modulating the activity of the NMDA receptors. For example, Rivat et al. (2008) have shown that a polyamine-free food diet for 7 days could reduce painful hypersensitivity in animal models of mono-arthritis (tibiotarsial injection of Freund's adjuvant) and peripheral mononeuropathy (loose ligature around the sciatic nerve) (Rivat et al. 2008).

The use of food compositions with low polyamines content intended to combat sensitization, the memorizing and chronicizing of pain, and more particularly pain of inflammatory origin, surgical-type incisional pain, or pain of monoarthritis origin involving the NR2-B subunit of the NMDA receptor, is known from document EP 1 648 431.

The combination of a food diet low in polyamines and the taking of docetaxel (Taxoter) has been described from the angle of therapeutic efficacy and the administering of analgesics (Cipolla et al, 2010).

Oxaliplatin however develops specific neurotoxicity in the two acute and chronic forms mentioned above, against which the current pharmacopeia proves to be practically powerless. There is therefore an extremely important need for a method for the specific treatment of neuropathic pain induced by platinum salts and more particular by oxaliplatin.

One first objective of the method is therefore to provide a method for the prevention and/or treatment of neuropathic pain induced by platinum salts and in particular by oxaliplatin.

A second objective of the invention is to provide the said prevention and/or treatment method having little or no side effects.

A third objective of the invention is to provide the said prevention and/or treatment method to limit the pauses, dose reductions or risks of chemotherapy withdrawal and therefore to promote the patient's chances of survival.

A fourth objective of the invention is to provide the said prevention and/or treatment method allowing the dose regimen to be increased or optimised via better management of the neurological adverse effects of these anticancer agents.

A fifth objective is to provide a method for treating cancer by chemotherapy, in some groups of patients.

Therefore the subject of the present invention is firstly a food composition for human or veterinary use, with low polyamines content, for use in the treatment and/or prevention of neuropathic pain induced by a platinum salt, in particular oxaliplatin.

A further subject is the use of a food composition with low polyamines content for the manufacture of a therapeutic food intended for the treatment and/or prevention of neuropathic pain induced by a platinum salt, in particular oxaliplatin. This use is intended for man or animal.

A further subject of is a method for treating and/or preventing of neuropathic pain induced by a platinum salt, in particular oxaliplatin, comprising the administration to a patient (human) or animal of a food composition or a therapeutic food with low polyamines content.

According to one characteristic, the use of the food composition or therapeutic food in the patient or animal consuming the same is accompanied by:
a reduction in excitatory glutamatergic transmission in the dorsal horn of the spinal cord; and/or
a reduction in tissue concentrations of glutamate and/or of its glutamine storage form in the dorsal horn of the spinal cord.

A further subject of the invention is a food composition with low polyamines content for human or veterinary use for use for inducing in a patient or animal consuming the same composition, a reduction in excitatory glutamatergic transmission in the dorsal horn of the spinal cord, and/or a reduction in the tissue concentrations of glutamate and/or of its glutamine storage form in the dorsal horn of the spinal cord. This use induces a reduction, suppression and/or prevention of neuropathic pain, in particular iatrogenic pain. The invention also concerns the method for treating and/or preventing neuropathic pain, in particular iatrogenic pain, comprising the administration of a food composition with low polyamines content intended to produce these effects. In particular, the targeted patient or animal is a patient or animal treated or will be treated with an anticancer agent, in particular a platinum salt, and preferably oxaliplatin. This method is intended to reduce, suppress and/or prevent neuropathic pain, in particular medication-induced, in particular such as pain induced by an anticancer agent, notably a platinum salt, more particularly oxaliplatin.

The composition or method of the invention may be used in the treatment and/or prevention of acute neuropathic pain induced by a platinum salt known to induce such pain, and more particularly oxaliplatin.

The composition or method of the invention may also be used in the treatment and/or prevention of chronic neuropathic pain induced by a platinum salt known to induce such pain.

By platinum salt known to induce chronic neuropathic pain is meant platinum salts essentially or exclusively inducing chronic neuropathic pain such as cisplatin, but also platinum salts not exclusively inducing chronic neuropathic pain such as oxaliplatin.

The composition or method of the invention may also be used in the treatment and/or prevention of acute or chronic neuropathic pain induced by a platinum salt known to induce such pain, such as oxaliplatin.

In one embodiment of the invention, the treatment with oxaliplatin comprises the administration of oxaliplatin in combination with 5-fluoro-uracil and folic acid.

This food composition or therapeutic food is composed in particular of a nutrient mixture comprising less than 1600 picomoles/g of polyamines relative to the weight of the composition, notably a content less than or equal to 1000, in particular less than or equal to 600, more particularly less than or equal to 400 and preferably less than or equal to 200 picomoles/g.

By veterinary use is meant the application of the invention to an animal. The animals concerned by the invention are more particularly pets, in particular dogs and cats, and racing animals particularly horses.

By polyamines is meant any organic compound having two or more amine functions and which have biological activity.

By <<biologically active polyamines>> is meant polyamines which have an effect on:
DNA stabilisation, condensation and conformation (Thomas et al (2001));
RNA transcription;
cell growth and proliferation by acting directly on cell cellular cycles (Thomas et al 2001);
regulation of immune response (Soulet D et al (2003)),
modulation of the functioning of the N-methyl-D-aspartate receptors (NMDA) which are involved in the process of neurodegeneration (Soulet et al Rivest (2003)).

By polyamines is more particularly meant putrescine, spermidine, spermine and cadaverine.

In one embodiment, the polyamines are present in the composition of the invention without being chelated, protected or masked.

By induced neuropathic pain is meant pain whose onset is the direct consequence of a lesion or pathology affecting the somatosensory system (Treede et al. 2008) caused by the administration of an anticancer agent, and notably associated with acute and chronic nociceptive disorders, allodynia and mechanical hyperalgesia and thermal hypersensitivity to cold.

In one embodiment of the invention, the nutrient mixture does not comprise any polyamines.

According to the invention, the nutrient mixture in the food composition comprises less than 400 picomoles/g of putrescine, less than 400 picomoles/g of spermidine, less than 400 picomoles/g of spermine and less than 400 picomoles/g of cadaverine, preferably less than 100 picomoles/g of putrescine, less than 100 picomoles/g of spermidine, less than 100 picomoles/g of spermine and less than 100 picomoles/g of cadaverine.

A normal food diet providing a daily intake of 2000 kcal (kilocalories), for an adult weighing 70 kg, depending on the food eaten, may comprise 200 000 to 700 000 nmol of polyamines per day, i.e. 100 to 350 nmol polyamines per kilocalorie per day.

A standard diet provides an intake of 250 nmol of polyamines per kilocalorie per day (Bardocz et al., (1995)).

To be able to convert quantities expressed in nanomoles of polyamines per kilocalorie into grams of polyamines per kilocalorie, it is necessary to consider a mean molecular weight for all the polyamines. This mean molecular weight of polyamines is a necessary approximation to perform this calculation and it is estimated at 145.24 g/mol.

In one embodiment of the invention, the nutrient mixture present in the food composition comprises per gram of composition less than 300, preferably less than 200 picomoles of putrescine, less than 50, preferably less than 20 picomoles of spermine, less than 150, preferably less than 100 picomoles of spermidine, less than 100, preferably less than 80 picomoles of cadaverine.

In one particular embodiment of the invention, the nutrient mixture in the food composition comprises per gram of composition less than 200 picomoles of putrescine, less than 20 picomoles of spermine, less than 100 picomoles of spermidine, less than 80 picomoles of cadaverine.

In another embodiment of the invention, the nutrient mixture in the food composition comprises less than 400 picomoles/g of spermidine, preferably less than 100 picomoles/g of spermidine.

In one particular embodiment, the composition does not comprise any spermidine.

The polyamines in the body are derived from three main sources: cell proliferation (physiological and tumour), food and intestinal bacteria. For best controlling of polyamines uptake in the body, it may be necessary to limit not only the exogenous intake via a perfectly controlled diet, but also to reduce the intracellular uptake of polyamines in particular by inhibiting endogenous synthesis of polyamines of cell origin or by blocking the transport of polyamines which takes place between the cell and the extracellular medium.

The endogenous synthesis of polyamines is based on the use of specific inhibitors. By <<specific inhibitor>> is designated a molecule capable of blocking in full or in part, directly or indirectly, whether or not reversibly, the active site of at least one of the enzymes involved in the synthesis of polyamines (ornithine decarboxylase-ODC, spermidine-spermine N1-acetyltransferase or spermine oxidase). The role of the inhibitor in the biosynthesis of polyamines is to stop or reduce significantly the endogenous production of polyamines in the body treated with the product of the present invention. The combined use of a polyamine synthesis inhibitor and a food intake low in polyamines allows a reduction in the quantity of bioavailable polyamines in the body.

Therefore, according to the invention, the food composition may comprise at least one inhibitor of the endogenous synthesis of polyamines.

In one embodiment of the invention, the food composition comprises a quantity of at least one inhibitor of polyamines synthesis corresponding to a daily dose, dependent on patient weight, of polyamines biosynthesis inhibitor(s) of about 5 to 20 mg/kg day, in particular 7 to 14 mg/kg/day, and more particularly about 9 mg/kg/day. These doses are given for a human being weighing about 70 kg.

For an animal, the daily dose of polyamines synthesis inhibitor(s) corresponds to about 2 to 10 g/kg/day, in particular 3 to 5 g/kg/day (Quenemer et al. (1995), Leveque et al (2000)).

In one embodiment of the invention, the food composition comprises at least one inhibitor of the intracellular synthesis of polyamines, in particular an inhibitor of ornithine decarboxylase, of spermidine-spermine N1-acetyltransferase or spermine oxidase, in particular in a proportion of no more than 15% by weight relative to the total dry weight of the composition.

In one particular embodiment of the invention, the inhibitor of intracellular polyamine synthesis is an inhibitor of ornithine decarboxylase, of spermidine-spermine N1-acetyltransferase or spermine oxidase.

Among the inhibitors of ODC, mention can be made of alpha-difluoromethylornithine (α-DFMO)

Other compounds able to inhibit ornithine decarboxylase, spermidine-spermine N1-acetyltransferase or spermine oxidase can be used. The quantities of inhibitor are adapted by persons skilled in the art on the basis of data on the biological activity of these compounds and their general knowledge.

In another embodiment of the invention, the food composition comprises at least one inhibitor of polyamines transport, in particular in a proportion of no more than 15% by weight relative to the total dry weight of the composition.

The transport of polyamines between the cell and the extracellular medium also allows fine-tuned adjustment of the intracellular content of polyamines. (Igarashi et al (2010)).

As polyamines transport inhibitor, mention can be made of different classes of molecules, in particular analogues of spermine (Burns (2009)) or dimers of polyamines (US 2005/0267220 A1), optionally bonded to an anthracene nucleus (WO 2010/148390). Therefore, the combined use of a polyamine transport inhibitor and a food intake low in polyamines allows a reduction in the quantity of bioavailable polyamines in the body.

For the purpose of reinforcing the reduction in endogenous synthesis of polyamines, it may be envisaged to have recourse to antibiotics to limit polyamines uptake through bacteria of intestinal flora.

In one embodiment of the invention, the food composition may comprise at least one antibiotic.

The use of antibiotics may lead to reducing the intake of vitamins, in particular those derived from the intestinal flora of patients. In this case, it may prove to be necessary to complete the composition with vitamins to prevent the onset of vitamin deficiency in patients in the event of extended administration of the composition.

By <<deficiency>> is designated lack of nutrients possibly deteriorating a patient's or animal's physical or mental condition.

Therefore, in one embodiment of the invention, the food composition may comprise vitamins.

In one particular embodiment of the invention, the composition comprises at least one antibiotic and/or is enriched with vitamins.

According to the invention, the nutrient mixture comprises at least one natural substance, at least one synthetic composition and/or a mixture thereof.

Advantageously, the natural substance of the invention is chosen from among foods for human or veterinary use.

According to the invention, the natural foods are chosen following the recommendations set by the nutritional guide for a food diet low in polyamines published by NUTRIALYS (www.guerir.org/magazine/guide-nutritionnel-nutrialys).

According to the invention, the synthetic composition is chosen from among all synthetic compositions whose polyamines content conforms to the invention and is less than 1600 picomoles/g of composition.

The synthetic composition of the invention may in particular be chosen from among the food compositions described in documents EP 0 703 731 and EP 1 648 431.

The synthetic composition of the invention may also be chosen from among POLYDOL® and CASTASE® products marketed by NUTRIALYS.

The composition or food with low polyamines content according to the invention is administered to the patient or animal, before, simultaneously with or after the treatment with a platinum salt, in particular oxaliplatin.

Advantageously, the composition or food with low polyamines content according to the invention is administered before and/or simultaneously with the treatment with the platinum salt, so as to maintain the content of exogenous polyamines at the lowest level possible during the treatment phase with the platinum salt, in particular oxaliplatin.

Preferably, the composition or food with low polyamines content according to the invention is therefore administered before and simultaneously with the treatment with the platinum salt, in particular oxaliplatin.

The administration of the composition or food with low polyamines content according to the invention can be given over a period of between 1 and 15 days, before the start of the treatment with the platinum salt, in particular oxaliplatin. This period is advantageously comprised between 5 and 10 days, typically it is 1 week. This administration is preferably maintained throughout the entire treatment with the platinum salt. This prior and/or simultaneous administration is advantageously repeated on each new administration of the platinum salt to the patient or animal.

The use of a polyamines-deficient food diet may comprise several phases during which the exogenous supply of polyamines is:
fully supplied by the food composition of the invention;
mostly supplied by the food composition of the invention;
partly supplied by the food composition of the invention.

By <<fully>> is meant the fact that the patient's food intake is limited to the compositions of the invention. No food other than the compositions of the invention is included in the patient's food diet. During this phase, polyamine depletion is maximal.

By <<mostly>> is meant the possibility of including in the patient's food diet a breakfast containing foods low in polyamines content. The remainder of the daily food ration is supplied by the compositions of the invention.

By <<partly>> is meant the possibility of including in the patient's food diet a breakfast and at least one solid meal comprising foods low in polyamines content. The remainder of the daily food ration is supplied by the compositions of the invention.

According to one variant of the invention, the composition or food low in polyamines content forms the entirety of the daily food ration of a human being or animal.

According to one variant of the invention, the composition or food with low polyamines content represents part of the daily food ration of a human being or animal, the said ration also comprising carbohydrates, lipids, proteins, vitamins, minerals and electrolytes in sufficient quantities to meet the daily nutritional needs of a human being or animal. One example of a ration including adequate quantities of carbohydrates, lipids, proteins, vitamins, minerals and electrolytes is described in document EP 0 703 731.

In one embodiment of the invention, the carbohydrates belong to the group comprising polymer of glucose, maltodextrins, sucrose, modified starches, glucose monohydrate, dehydrated glucose syrup, glycerol monostearate and mixtures thereof.

In one embodiment of the invention, the proteins belong to the group comprising soluble milk proteins, soy proteins, serum peptides powdered egg white, potassium caseinate, non-phosphorylated peptides, casein peptides, mixed caseinate, soybean isolate and mixtures thereof.

In one embodiment of the invention, the lipids of the composition used belong to the group comprising butter oil, groundnut oil, medium chain triglycerides, grape seed oil, soybean oil, evening primrose oil and mixtures thereof.

In one particular embodiment of the invention, the lipids consist of a mixture of at least one oil of animal origin, at least one oil of vegetable origin and glycerol stearate.

If the patient is a human being, the food composition of the invention may form the daily food ration of a human being and comprise:
75 g to 500 g of carbohydrates,
20 g to 185 g of lipids,
20 g to 225 g of proteins,
vitamins, minerals and electrolytes in sufficient quantities to meet the daily nutritional needs of a human being;
and optionally an inhibitor of polyamines intracellular synthesis in the proportion of less than 50 g and preferably a proportion of 0.3 to 10 g per day.

In one embodiment of the invention, the food composition represents a sub-multiple of a daily food ration of a human being and comprises:
75/X g to 500/X g of carbohydrates;
20/X g to 185/X g of lipids;
20/X g to 225/X g of proteins;
vitamins, minerals and electrolytes in sufficient quantities for partial meeting of the daily nutritional needs of a human being,
and optionally an inhibitor of intracellular polyamines synthesis in a proportion of less than 50/X g and preferably in a proportion of 0.3/X to 10/X g per day,
   X being an integer of between 2 and 8 and corresponding to the number of rations to be ingested by the patient to meet daily nutritional needs.

For an animal, the daily food ration is adapted in relation with the category and weight of the animal, whether a pet or racing animal. The distribution of carbohydrates, lipids and proteins and the needs for vitamins, minerals and electrolytes of an animal's daily food ration are well known. Regarding the inhibitor of intracellular polyamines synthesis, the dose is adapted in relation with the animal's weight, optionally on the basis of data obtained from man.

Therefore, the food composition of the invention may form the daily food ration or a sub-multiple of a daily food ration of an animal and must meet an animal's daily nutritional needs.

In one embodiment of the invention, the total quantity of polyamines ingested per day by a patient does not exceed 0.40 nanomoles per kcal of ingested composition, in particular 0.30 nanomoles per kcal of ingested composition, more particularly 0.25 nanomoles per kcal of ingested composition and most particularly 0.20 nanomoles per kcal of ingested composition.

In another embodiment of the invention, polyamines are present in the composition of the invention at a level 100 times lower, particularly 500 times lower, more particularly 1000 times lower than the quantity of polyamines present in a normal food diet.

A further subject of the invention concerns a platinum salt, in particular oxaliplatin, for its use in the treatment of cancer in patients or animals receiving or having received a food diet with low polyamines content. A further subject of the invention is the use of a platinum salt, in particular oxaliplatin, for the treatment of cancer in patients or animals receiving and/or having received a food diet with low polyamines content.

Advantageously, the platinum salt, in particular oxaliplatin, is used in the treatment of cancer in patients or animals having received and still receiving a food diet with low polyamines content.

In one embodiment of the invention, the platinum salt, in particular oxaliplatin, is used in the treatment of cancer in patients or animals having received or still receiving a food diet with low or no spermidine content.

A further subject of the invention is a method for the anti-cancer treatment in which a platinum salt is administered, in particular oxaliplatin, to a patient or animal receiving and/or having received a food diet with low polyamines content.

Advantageously, the method for the treatment comprises the administration of a platinum salt, in particular oxaliplatin, to a patient or animal having received and still receiving a food diet with low polyamines content.

In one embodiment of the invention, the method for the anti-cancer treatment comprises the administration of a platinum salt, in particular oxaliplatin, to a patient or animal having received and still receiving a food diet with low or no spermidine content.

The patient or animal, receiving and/or having received a food diet with low polyamines content, consumes or has consumed a food composition or a therapeutic food of the invention. Here all the characteristics and procedures for use of the compositions and therapeutic foods defined above are applicable.

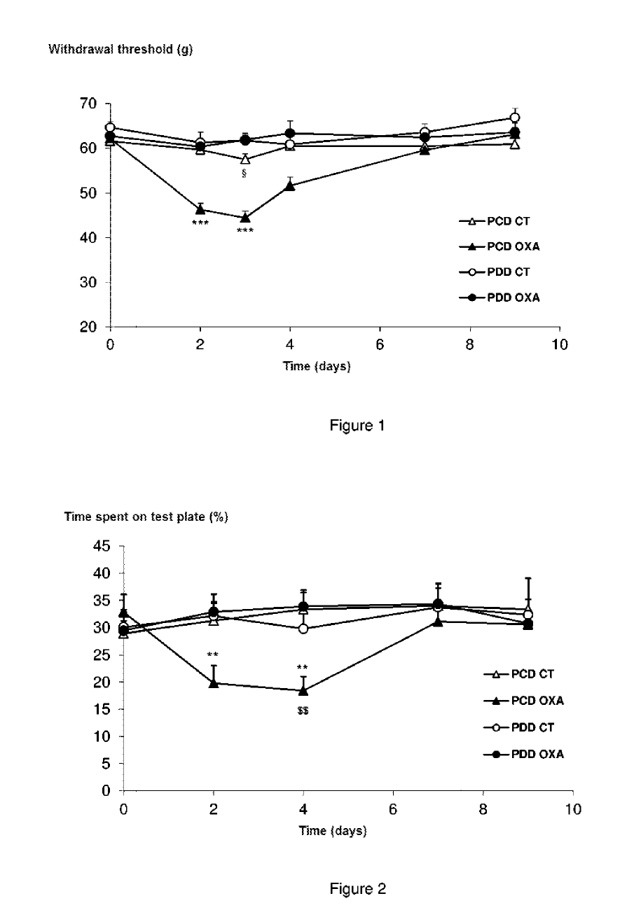

FIG. 1 shows the effect of a diet based on a composition with low polyamines content on mechanical hypersensitivity induced by oxaliplatin.

FIG. 2 shows the effect of a diet based on a composition with low polyamines content on thermal hypersensitivity induced by oxaliplatin.

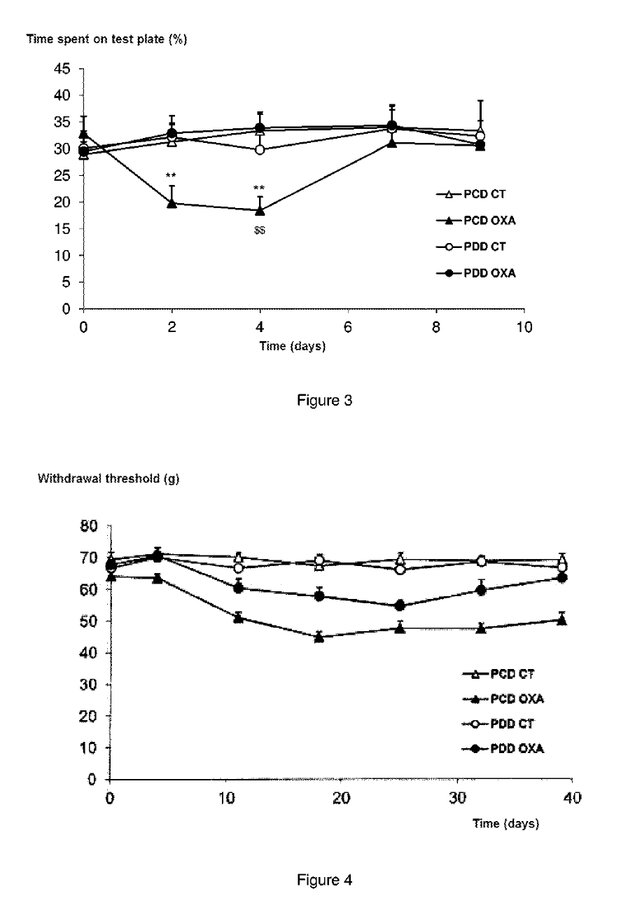

FIG. 3 shows the effect of a diet based on a composition with low polyamines content on thermal hypersensitivity induced by oxaliplatin.

FIG. 4 shows the effect of a diet based on a composition with low polyamines content on mechanical hypersensitivity induced by repeated injections of oxaliplatin.

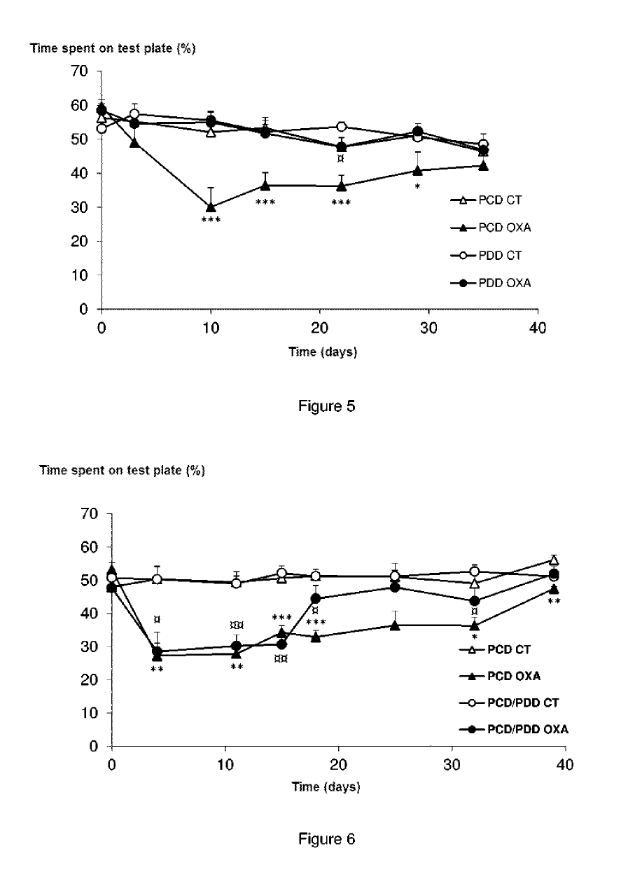

FIG. 5 shows the effect of a diet based on a composition with low polyamines content on hypersensitivity to cold induced by repeated injections of oxaliplatin.

FIG. 6 shows the effect of a diet based on a composition with low polyamines content on hypersensitivity to cold induced by repeated injections of oxaliplatin.

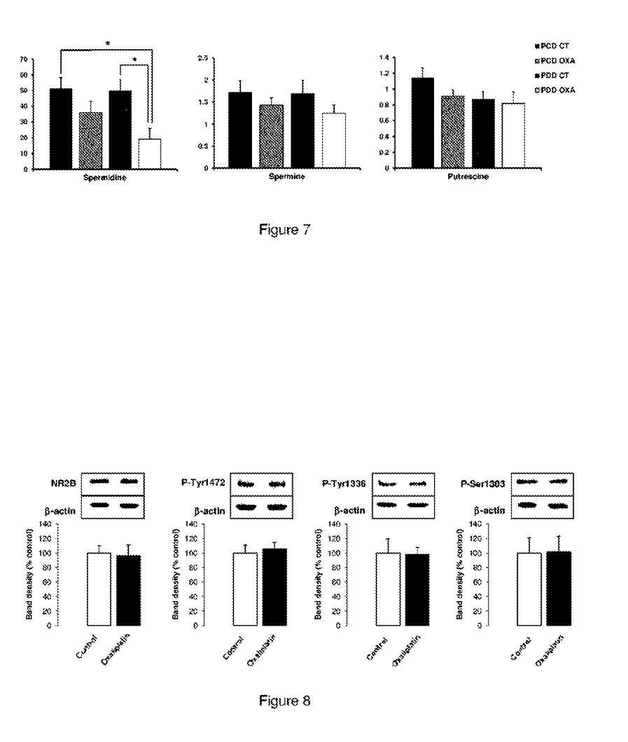

FIG. 7 shows the effect of a diet, with low polyamines content on the level of erythrocyte polyamines.

FIG. 8 gives the measurement of the expression of NR2B subunit and of phosphorylation of NR2B subunit of the NMDA receptors in the acute neuropathy model induced by oxaliplatin.

The different subject-matters of the invention and their embodiments will be better understood on reading the following Examples. These Examples are given by way of indication and are non-limiting.

### EXAMPLES

For each of the following Examples:

The experiments were conducted on male Sprague-Dawley rats weighing 150 to 175 grams at the start of the study (Charles River, Saint-Aubin-lés-Elbeuf, France). The rats were distributed among plastic cages with 4 rats per cage. The cages were placed in an air-conditioned room at 22° C.±2° C., with controlled hygrometry (50%), a day/night 12-hour lighting cycle, and free access to food and water.

The oxaliplatin (Merck®, France) was reconstituted in 5% glucose solution (Freeflex®, Fresenius Kabi, France) at a concentration of 1 mg/mL to obtain an injectable volume of 6 mL/kg via intraperitoneal route and 2 mL/kg via intravenous route.

Two types of compositions were used:
a composition containing a quantity of polyamines equivalent to that present in a daily food diet (Putrescine: 54 mg/kg, Cadaverine: 37 mg/kg, Spermidine: 27 mg/kg, Spermine: 7 mg/kg),
a composition with low polyamine content comprising less than 1600 picomoles/g polyamines (Putrescine<0.01 mg/kg, Cadaverine<0.01 mg/kg, Spermidine<0.01 mg/kg, Spermine<0.01 mg/kg).

The different types of diets (diet based on a composition comprising a normal content of polyamines or PCD (Polyamine Containing Diet) and a diet based on a composition with low polyamines content called PDD (Polyamine Deficient Diet)) were given as soon as the rats arrived in the animal housing facility i.e. one week before the start of experiments. The treatments (oxaliplatin or glucose 5%) were randomised in each cage, i.e. 2 animals treated with oxaliplatin and 2 control animals per cage.

The rats were allowed one week of attunement before the start of experiments. During this period of acclimatisation, the rats were accustomed to the housing conditions and to handling by the person conducting the tests. For the Thermal Place Preference Test, the rats were placed under reversed cyclic lighting to obtain maximum rat exploring capacity during experiments which were conducted in the daytime.

All the behaviour tests were blind tests relative to the treatment (oxaliplatin or vehicle) and to diet (normal polyamines content or polyamines-deficient).

Two models of animals with oxaliplatin-induced neuropathy were used:

For the animal model of acute neuropathy induced by oxaliplatin in rat: nociceptive disorders were induced by a single administration of oxaliplatin (OXA) via intraperitoneal route, at a dose of 6 mg/kg. The animals of the control group (CT) had an injection of the vehicle (5% glucose solution) (Ling et al. 2007b).

For the animal model of chronic neuropathy induced by oxaliplatin in rat: oxaliplatin (OXA) is administered via intravenous route at a dose of 2 mg/kg. The animals of the control group (CT) had an injection of the vehicle (5% glucose solution). The injections were given on D0-4-7-11-14-18-21-25, i.e. an accumulated dose of 16 mg/kg over 4 weeks (Ling et al. 2007a).

The following behaviour tests were used:

Electronic Von Frey Test

The electronic von Frey electronic apparatus (Bioseb®, Chaville, France) has a hand-held probe formed of a tip in plastic connected to a force sensor. The rats are placed in bottom-less Plexiglas boxes (30 cm×30 cm×25 cm) arranged on a raised grid support. After a rat habituation time of 15 minutes, the tip is applied perpendicular to the center of the 5 plantar pads of each hind paw. The pressure applied is gradually increased until it causes flexion reflex of the hind paw. The intensity of the stimulus, maximum applied pressure (expressed in grams) is automatically recorded by the apparatus when the paw is withdrawn. The value corresponds to the animal's mechanical nociceptive threshold (allodynia/hyperalgesia). Three measurements are taken with a minimum interval time of 15 minutes. The calculation of the mean and standard deviation of the 3 measurements is then determined.

Thermal Place Preference Test

The Thermal Place Preference Test (Bioseb®, Chaville, France) is formed of two adjacent metal plates surrounded by a chamber in opaque Plexiglas. The rat is placed in the chamber and is free to move from one plate to the other. The first plate called the <<reference plate>> is held at ambient temperature (25-30° C.) and the second plate called the <<test plate>> is held at a cold temperature. Using an infrared camera connected to a computer, the time that the animal spends on each plate is measured, together with the number of movements between the two plates. Each session lasts 3 minutes. Habituation of the animals consists of placing the rats on the apparatus for 3 minutes several times a day maintaining the two plates at a temperature of 25° C. Under these conditions, the rats spend the same amount of time on each plate and do not preferred any place. Three pairs of temperatures were chosen for the experiments in relation to the model examined:

Acute neuropathy oxaliplatin model: 12° C. vs 25° C. et 19° C. vs. 25° C.

Chronic neuropathy oxaliplatin model: 20° C. vs. 30° C.

To assess the preventive impact of a diet based on a composition with low polyamines content on acute neuropathy induced by oxaliplatin: the injection of oxaliplatin was given on D0, i.e. one week after the start of the PCD or PDD diets. The nociception tests were performed before inducing nociceptive disorders via oxaliplatin injection (D0, base thresholds) then repeated on D2, D3, D4, D7 and D9. The Open Field test was conducted 3 days after the injection of oxaliplatin i.e. at the peak of the nociceptive disorders.

To assess the metabolic variations induced by a diet based on a composition with low polyamines content in the dorsal horn of the spinal cord by proton NMR spectroscopy, the nociceptive thresholds were determined just before sacrificing the animals by decapitation. The spinal cord of the rats was then quickly removed to isolate the dorsal horn from L4 to L6. The samples were taken on D3 (peak of nociceptive disorders) and D9 (return of nociceptive thresholds to their normal value). Each tissue sample taken was immediately immersed in liquid nitrogen and stored at −80° C. until analysis.

Extraction of the polar metabolites was performed using the following protocol: to obtain a sufficient quantity of tissue for analysis by ¹H-NMR spectroscopy, the spinal cord tissue samples taken from 2 rats which had received the same treatment conditions were combined. Extraction of the metabolites was performed following the protocol described by Angenstein et al. (2008) and the recommendations of Beckonert et al. (2007) (Beckonert et al. 2007; Angenstein et al. 2008). All the steps of the protocol were conducted on ice or at 4° C. The sampled tissues were placed in 1.5 mL Eppendorf tubes. A volume of 5 mL of 6% perchloric acid (stored at 4° C.) per gram of tissue was added to each tube. The tissues were then roughly cut up using scissors. With 6% perchloric acid it is possible to dissolve the tissues and extract the metabolites. After ultrasonication, the sample was vortexed for 30 seconds and then centrifuged at 14000 rpm for 15 minutes. The supernatant was collected and the pH adjusted to 11 with 10% KOH solution. Centrifuging at 14000 rpm for 10 minutes allowed removal of the precipitate of potassium perchlorate. The supernatant containing the extracted tissue metabolites was frozen to −80° C. then lyophilised overnight. Before ¹H-NMR spectroscopic analysis, the lyophilised sample was reconstituted with 600 μL of deuterated water (D₂O) containing 1 mM sodium salt of 2,2,3,3-d4 3-(Trimethylsilyl)propionic acid (TSP-d4, Sigma Aldrich). The TSP-d4 was used as internal concentration reference and its chemical shift on each spectrum acquired by ¹H-NMR is 0 ppm.

The NMR spectra were then acquired as follows: one-dimensional spectra acquisition (1D) was performed on a Brucker AC 400 Mhz NMR spectrometer (NMR technical platform, Ecole Nationale Supérieure de Chimie in Clermont-Ferrand). Before each acquisition, pre-saturation was programmed to attenuate the signal of residual water present in the sample. Similarly, the number of scans (ns) or number of accumulations throughout acquisition was set at 32. These two conditions (pre-saturation and ns=32) allowed an improved signal-to-noise ratio. Each metabolite present in a given tissue extract was able to be identified in the 1D ¹H-NMR spectrum by its chemical shift (expressed in ppm), by means of specific databases (Fan 1996; Govindaraju et al. 2000; http://www.hmdb.ca). The concentration of each metabolite identified in the 1D spectrum was calculated. The area under each peak allocated to a metabolite of interest was measured using spectra processing software (TopSpin™, Brucker), and this area was directly compared with that of TSP-d4 whose concentration was initially set at 1 mM. The concentration of each metabolite of interest was therefore able to be calculated, expressed in mol/g of tissue. This analytical technique allowed the identification and quantification of 18 metabolites extracted from the dorsal horn of the spinal cord (Table 1).

TABLE 1

| | | Abbrevia- | Chemical |
|---|---|---|---|
| Class | Metabolite | tion | shift (ppm) |
| Bioenergetic | Adenosine phosphates | AXP | 8.28-8.25 |
| derivatives | Lactate | Lac | 1.36-1.30 |
| | Acetate | Ace | 1.93-1.91 |
| | Myoinositol | Myl | 3.66-3.58 |
| | Alanine | Ala | 1.52-1.43 |
| | β-hydroxybutyrate | BHB | 1.24-1.19 |
| | Total creatine | tCr | 3.04-3.01 |
| | Succinate | Succ | 2.415-2.395 |
| Neurotransmitters | γ-aminobutyric acid | GABA | 2.24-2.17 |
| and derivatives | Glutamate | Glu | 2.34-2.26 |
| | Glutamine | Gln | 3.80-3.74 |
| | Aspartate | Asp | 2.70-2.66 |
| | N-acetylaspartate | NAA | 2.04-2.00 |
| | N-acetylaspartylglutamate | NAAG | 2.07-2.04 |
| Lipid and | Choline | Cho | 3.21-3.185 |
| phospholipid | Phosphocholine | PC | 3.24-3.22 |
| derivatives | Phosphatidylcholine | PtC | 3.29-3.26 |
| | Glycerophosphocholine | GPC | 3.31-3.29 |

The mean concentrations and standard deviations are given in histogram form in percentage of the control group (non-treated rats on a polyamine-containing diet, PCD CT).

To assess the preventive impact of a diet based on a composition with low polyamines content on chronic neuropathy induced by oxaliplatin: as soon as they entered the animal housing, a PDD diet was given for one half of the cages. The nociception tests were performed before inducing neuropathy (D0, base thresholds), once a week during the injections and after withdrawal of treatment for 15 days (at D0, D2, D8, D16, D23, D30 and D36). The Open Field test was performed on D31.

To evaluate the curative impact of a diet based on a composition with low polyamines content on chronic neuropathy induced by oxaliplatin: all the rats were initially submit to a normal diet (PCD) one week before the start of experiments. Once neuropathy was well established (D15), one half of the rats were fed a PDD diet until the end of the study. The nociception tests were performed before inducing neuropathy (D0, base thresholds), once a week during the injections and after the withdrawal of treatment for 15 days (at D0, D4, D11, D15, D18, D25, D32 and D39).

Statistical analysis of data was determined using STATA v10 software (StataCorp). Variance analysis (ANOVA) followed by a post hoc Tuckey Kramer test were carried out to compare the results of the different groups. To compare the groups 2 by 2 at each time, a Kruskal-Wallis test was performed with Bonferroni correction.

For the metabolomic study of the dorsal corn of the spinal cord, PLS analysis (partial least squares regression) was performed on spectra data using Simca-P+12 software (Umetrics). PLS analysis allowed classification of the metabolites which contributed most to discrimination between the different treatment groups. VIP (variable importance in the projection) was calculated for each metabolite and was used as criterion for its discriminatory value (Chong et al. 2005). The limit value of VIP was chosen to evidence the 5 most discriminating metabolites between 2 different groups.

### Example 1

### Evaluation of the Preventive Impact of a Diet Based on a Composition with Low Polyamines Content on Acute Neuropathy Induced by Oxaliplatin Using the Electronic Von Frey Test (FIG. 1)

The results show that mechanical hypersensitivity is observed 2 days after oxaliplatin injection in rats subjected to a PCD diet (PCD OXA). This hypersensitivity persists for 4 days and disappears completely on the 7^{th} day after oxaliplatin injection. In rats treated with oxaliplatin given a PDD diet (PDD OXA), no variation in mechanical hypersensitivity could be highlighted. The exogenous depletion of polyamines allows the suppressing of mechanical allodynia/hyperalgesia induced by oxaliplatin but does not generate an analgesic effect. Indeed, no difference is seen at the mechanical thresholds between control animals given a PDD diet (PDD CT) and control animals given a PCD diet (PCD CT).

### Example 2

### Evaluation of the Preventive Impact of a Diet Based on a Composition with Low Polyamines Content on Acute Neuropathy Induced by Oxaliplatin Using the Thermal Place Preference Test 12° C. vs. 25° C. (FIG. 2)

The results show that the rats treated with oxaliplatin and given a PDD diet spend on average as much time on the test plate as the control animals, showing no hypersensitivity to cold.

### Example 3

### Evaluation of the Preventive Impact of a Diet Based on a Composition with Low Polyamines Content on Acute Neuropathy Induced by Oxaliplatin Using the Thermal Place Preference Test 19° C. Vs. 25° C. (FIG. 3)

The results show that the rats treated with oxaliplatin and given a PDD diet spend on average as much time on the test plate as the control animals, not showing any hypersensitivity to cold.

### Example 4

### Preventive Effect of a Diet Based on a Composition with Low Polyamines Content on Chronic Neuropathy Induced by Oxaliplatin Using the Electronic Von Frey Test (FIG. 4)

The results show that the mechanical hypersensitivity developed in animals on a PDD diet is less intense than that developed in rodents given a PCD diet; this difference occurring as early as D4 (−10%; p<0.05), reaching a maximum at D11 (−15%; p<0.01) and persisting until the end of treatment at D39 (−21%; p<0.01).

The mechanical hypersensitivity developed in animals on a PDD diet is therefore less intense than that developed in rodents on a PCD diet.

### Example 5

### Preventive Effect of a Diet Based on a Composition with Low Polyamines Content on Hypersensitivity to Cold Induced by Repeated Injections of Oxaliplatin Using the Thermal Place Preference Test 20° C. vs. 30° C. (FIG. 5)

No difference was observed in the time spent on the test plate between the two groups of control rats given a PCD or PDD diet.

Right from the first injection of oxaliplatin, a drop was observed in the time spent on the test plate (20° C.) compared with the reference plate (30° C.) for the group of rats treated with oxaliplatin compared with the group of control rats given a PCD diet. This reduction in the time spent on the test plate became significant after the 3^{rd} injection of oxaliplatin i.e. at D10. The difference is maximal at D10 (−42%; p<0.001) then increases at D15 and D22 (−32%; p<0.001 and −24%; p<0.001, respectively). At D29, the decrease becomes weakly significant (−20%; p<0.05).

For the group of rats treated with oxaliplatin given a PDD diet, at D22 after the start of injections a weakly significant difference was observed (−11%; p<0.05) compared with the group of control rats given a PDD diet.

The results show that a diet with low polyamines content allows complete prevention of the onset of hypersensitivity to cold. In addition, the introduction of a diet low polyamines content after the onset of neuropathy fully reverses this thermal hypersensitivity.

### Example 6

### Curative Effect of a Diet Based on a Composition with Low Polyamines Content on Hypersensitivity to Cold Induced by Repeated Injections of Oxaliplatin (FIG. 6)

No variation was observed in the time spent on the test plate between the two groups of control rats on a PCD or PDD diet.

For the group of rats treated with oxaliplatin and given a PCD diet, a drop was observed in the time spent on the test plate (20° C.) compared with the reference plate (30° C.) right after the first injection of oxaliplatin compared with the group of control rats. This reduction in the time spent on the plate became significant on and after the 2^{nd} injection of oxaliplatin i.e. at D4. The difference is maximal at D4 (−46%; p<0.01) and persists up until D39 (−15%; p<0.01).

For the group of rats treated with oxaliplatin and fed with a PDD diet started on D15, an increase was observed in the time spent on the test plate at D18 (+31%). As soon as the PDD diet was given, the thermal thresholds of the animals treated with oxaliplatin again become comparable to those of animals in the control group (−13%; p>0.05).

### Example 7

### Evaluation of Metabolic Variations Induced by a Diet Based on a Composition with Low Polyamines Content in the Dorsal Horn of the Spinal Cord Using Proton NMR Spectroscopy on Acute Neuropathy Induced by Oxaliplatin

The variations in the metabolites extracted from the dorsal horn of the spinal cord were measured by proton NMR spectroscopy on one-dimensional spectra (1D). The mean concentrations of each metabolite (expressed in mol/g of tissue) as a function of the different experimental conditions are given in Table 2 (D+3) and Table 3 (D+9).

TABLE 2

| Metabolites | PCD CT | PCD OXA | PDD CT | PDD OXA |
|---|---|---|---|---|
| AXP | 0.210 ± 0.015 | 0.168 ± 0.023 | 0.163 ± 0.016 | 0.142 ± 0.021 |
| Lac | 6.629 ± 0.481 | 5.718 ± 0.881 | 5.606 ± 0.278 | 4.787 ± 0.517 |
| Ace | 0.324 ± 0.063 | 0.206 ± 0.063 | 0.407 ± 0.168 | 0.331 ± 0.138 |
| Myl | 3.011 ± 0.220 | 2.629 ± 0.332 | 2.572 ± 0.229 | 2.289 ± 0.204 |
| Ala | 0.346 ± 0.143 | 0.365 ± 0.058 | 0.380 ± 0.045 | 0.307 ± 0.036 |
| BHB | 0.504 ± 0.085 | 0.502 ± 0.051 | 0.526 ± 0.056 | 0.492 ± 0.059 |
| tCr | 3.337 ± 0.256 | 2.635 ± 0.280 | 2.681 ± 0.199 | 2.517 ± 0.197 |
| Succ | 0.376 ± 0.097 | 0.711 ± 0.242 | 0.317 ± 0.019 | 0.292 ± 0.083 |
| GABA | 2.294 ± 0.098 | 2.069 ± 0.293 | 2.266 ± 0.112 | 1.838 ± 0.155 |
| Glu | 0.385 ± 0.240 | 0.711 ± 0.242 | 0.318 ± 0.019 | 0.292 ± 0.083 |
| Gln | 0.577 ± 0.196 | 0.578 ± 0.071 | 0.488 ± 0.050 | 0.483 ± 0.032 |
| Asp | 0.729 ± 0.262 | 1.011 ± 0.351 | 0.652 ± 0.116 | 0.651 ± 0.116 |
| NAA | 2435 ± 0.493 | 2.272 ± 0.371 | 2.140 ± 0.190 | 1.721 ± 0.156 |
| NAAG | 0.618 ± 0.174 | 0.568 ± 0.086 | 0.479 ± 0.042 | 0.433 ± 0.054 |
| Cho | 1.315 ± 0.065 | 1.225 ± 0.158 | 1.193 ± 0.070 | 1.077 ± 0.128 |
| PC | 1.024 ± 0.116 | 0.958 ± 0.103 | 0.931 ± 0.054 | 0.832 ± 0.081 |
| PtC | 0.730 ± 0.096 | 0.615 ± 0.057 | 0.549 ± 0.053 | 0.499 ± 0.035 |
| GPC | 0.345 ± 0.055 | 0.285 ± 0.014 | 0.267 ± 0.016 | 0.232 ± 0.025 |

TABLE 3

| Metabolite | PCD CT | PCD OXA | PDD CT | PDD OXA |
|---|---|---|---|---|
| AXP | 0.174 ± 0.031 | 0.185 ± 0.058 | 0.137 ± 0.033 | 0.161 ± 0.050 |
| Lac | 5.465 ± 0.735 | 4.789 ± 0.934 | 5.260 ± 0.184 | 5.266 ± 0.453 |
| Ace | 0.170 ± 0.022 | 0.142 ± 0.040 | 0.414 ± 0.146 | 0.324 ± 0.104 |
| Myl | 2.507 ± 0.128 | 2.702 ± 0.734 | 2.200 ± 0.181 | 2.215 ± 0.260 |
| Ala | 0.303 ± 0.025 | 0.430 ± 0.227 | 0.301 ± 0.063 | 0.357 ± 0.072 |
| BHB | 0.432 ± 0.051 | 0.340 ± 0.098 | 0.481 ± 0.027 | 0.453 ± 0.073 |
| tCr | 2.704 ± 0.489 | 2.461 ± 0.432 | 2.375 ± 0.530 | 2.509 ± 0.551 |
| Succ | 0.482 ± 0.156 | 0.343 ± 0.087 | 0.353 ± 0.084 | 0.280 ± 0.080 |
| GABA | 1.298 ± 0.704 | 1.577 ± 0.393 | 1.942 ± 0.242 | 1.617 ± 0.330 |
| Glu | 0.488 ± 0.245 | 0.440 ± 0.173 | 0.424 ± 0.175 | 0.517 ± 0.106 |
| Gln | 0.609 ± 0.157 | 0.498 ± 0.095 | 0.455 ± 0.109 | 0.195 ± 0.186 |
| Asp | 0.775 ± 0.192 | 0.658 ± 0.147 | 0.757 ± 0.112 | 0.637 ± 0.207 |
| NAA | 2.183 ± 0.260 | 2.040 ± 0.282 | 2.005 ± 0.079 | 1.887 ± 0.228 |
| NAAG | 0.512 ± 0.036 | 0.493 ± 0.132 | 0.467 ± 0.017 | 0.436 ± 0.119 |
| Cho | 0.899 ± 0.295 | 1.110 ± 0.291 | 1.180 ± 0.066 | 0.927 ± 0.142 |
| PC | 1.247 ± 0.352 | 0.837 ± 0.255 | 0.942 ± 0.117 | 0.899 ± 0.178 |
| PtC | 0.590 ± 0.109 | 0.591 ± 0.133 | 0.437 ± 0.106 | 0.487 ± 0.068 |
| GPC | 0.270 ± 0.026 | 0.293 ± 0.090 | 0.226 ± 0.052 | 0.209 ± 0.035 |

The integration of these metabolic variations in the different biochemical cellular pathways is interpreted in the discussion. The variations in concentrations of the metabolites were normalised in relation to the controls (Group 1).

Three days after the injection of oxaliplatin, the tissue concentrations of acetate (Ace, −36%, p<0.05) and adenosine phosphates (AXP, −20%, p<0.05) were significantly reduced in animals treated with oxaliplatin compared with the control animals which were given a food diet containing polyamines (Group 1 vs. Group 2) In the control rats given a diet based on a composition with low polyamines content, the tissue concentrations of acetate (Ace, −60%, p<0.05), of phosphatidylcholine (PtC, −24%, p<0.05), of glycerophosphocholine (GPC, −22%, p<0.05) and adenosine phosphates (AXP, −22%, p<0.05) decreased significantly compared with the control animals given a diet containing polyamines (Group 3 vs. Group 1). In the rats treated with oxaliplatin and given a diet based on a composition with low polyamines content, the tissue concentrations of GABA (−19%, p<0.05) and alanine (Ala, −11%, p<0.05) were significantly reduced compared with the control animals given a diet without polyamines (Group 4 vs. Group 3). In comparison, the tissue concentrations of glutamate (Glu, −59%, p<0.05), of glutamine (Gln, −16%, p<0.05), lactate (Lac, −16%, p<0.05), phosphatidylcholine (PtC, −19%, p<0.05), glycerophosphocholine (GPC, −19%, p<0.05) and adenosine phosphates (AXP, −15%, p<0.05) were significantly reduced in animals treated with oxaliplatin and given a diet based on a composition with low polyamines content compared with the animals treated with oxaliplatin and on a diet containing polyamines (Group 4 vs. Group 2).

Nine days after the injection of oxaliplatin, the tissue concentrations of phosphocholine (PC, −32%, p<0.05) were significantly reduced in rats treated with oxaliplatin compared with the control rats fed on a diet containing polyamines (Group 2 vs. Group 1). In the control rats given a diet based on a composition with low polyamines content, the tissue concentrations of phosphatidylcholine (PtC, −26%, p<0.05) and N-acetylaspartylglutamate (NAAG, −8%, p<0.05) were significantly reduced compared with the control rats receiving a polyamine-containing diet (Group 3 vs. Group 1). Conversely, the tissue concentrations of acetate (Ace, +143%, p<0.05) were significantly increased in control rats receiving a diet based on a composition with low polyamines content compared with the control rats on a diet containing polyamines (Group 3 vs. Group 1). In the rats treated with oxaliplatin and given a diet based on a composition with low polyamines content, the tissue concentrations of glutamine (Gln, −57%, p<0.05) and choline (Cho, −21%, p<0.05) showed a significant decrease compared with the animals in the control group given a diet without polyamines (Group 4 vs. Group 3). In the rats treated with oxaliplatin and given a diet based on a composition with low polyamines content, the tissue concentrations of glutamine (Gln, −60%, p<0.05) showed a significant decrease compared with the animals treated with oxaliplatin and on a diet containing polyamines (Group 4 vs. Group 2). Conversely, the tissue concentrations of acetate (Ace, +128%, p<0.05) and β-hydroxybutyrate (BHB, +33%, p<0.05) were significantly increased in the rats treated with oxaliplatin and receiving a diet based on a polyamine deficient composition compared with the animals treated with oxaliplatin and on a diet containing polyamines (Group 4 vs. Group 2).

The most discriminatory metabolites between the different groups identified by PLS analysis, are given in Tables 4 and 5.

TABLE 4
D + 3

| PCD CT vs. | | PCD CT | | PDD CT | | PDD OXA | |
|---|---|---|---|---|---|---|---|
| PCD OXA | | vs. PDD | | vs. PDD | | vs. PCD | |
| Metabo- | | CT | | OXA | | OXA | |
| lite | VIP | Metabolite | VIP | Metabolite | VIP | Metabolite | VIP |
| tCr | 1.415 | tCr | 1.364 | GABA | 1.610 | Ala | 1.558 |
| Ace | 1.365 | AXP | 1.333 | NAA | 1.505 | GPC | 1.224 |
| AXP | 1.324 | Lac | 1.259 | Lac | 1.393 | NAAG | 1.146 |
| Glu | 1.206 | BHB | 1.242 | Ala | 1.327 | PtC | 1.145 |
| GPC | 1.201 | PtC | 1.188 | GPC | 1.290 | Glu | 1.135 |

TABLE 5
D + 9

| PCD CT vs. | | | | | | | |
|---|---|---|---|---|---|---|---|
| PCD OXA | | PCD CT vs. | | PDD CT vs. | | PDD OXA vs. | |
| Metabo- | | PDD CT | | PDD OXA | | PCD OXA | |
| lite | VIP | Metabolite | VIP | Metabolite | VIP | Metabolite | VIP |
| Ace | 1.692 | Ace | 1.481 | Cho | 2.049 | Ace | 1.776 |
| tCr | 1.549 | Myl | 1.422 | Gln | 1.802 | Gln | 1.563 |
| GABA | 1.529 | NAAG | 1.309 | GABA | 1.400 | BHB | 1.420 |
| Lac | 1.508 | PtC | 1.222 | Succ | 1.159 | GPC | 1.236 |
| BHB | 1.372 | Cho | 1.169 | Ala | 1.097 | PtC | 1.103 |

Metabolome analysis of the dorsal horn of the spinal cord by proton NMR spectroscopy allowed the evidencing of significant variations in cell metabolites, in response to treatment with oxaliplatin and/or exogenous polyamine depletion.

At D+3, oxaliplatin induced an increase in concentrations of glutamate in the dorsal horn of the spinal cord in treated rats receiving a normal diet. Without being bound by any theory, this increase in glutamate may contribute towards transmission of the pain message causing nociceptive disorders induced by oxaliplatin. In the control rats receiving a food diet with low polyamines content, a reduction in glutamate was observed at D+3 and at D+9. In the rats treated with oxaliplatin and receiving a food diet with low polyamines content, the tissue concentration of glutamate was significantly reduced at D+3. One explanation of the preventive effect of the diet based on a composition with low polyamines content against the onset of nociceptive disorders induced by oxaliplatin could be the inhibition of excitatory glutamatergic transmission in the dorsal horn of the spinal cord, involving the NMDA receptors. The tissue concentrations of glutamine, a form of astrocyte glutamate storage (Zwingmann et al. 2005), were reduced at D+3 and D+9 in the spinal cord of rats treated with oxaliplatin and receiving a food diet with low polyamines content. On the other hand, these concentrations did not vary compared with those of the control animals at D+3 and D+9.

On the basis of these metabolic observations, it can strongly be envisaged that the combining of treatment with oxaliplatin and a diet based on a composition with low polyamines content leads to global slowing of the metabolism of the neurons in the dorsal horn of the spinal cord, at glutamatergic transmission level. These metabolic perturbations are not generated by lack of exogenous glucose intake, the major energy substrate of cells, since the levels of β-hydroxybutyrate, a ketone body consumed in the event of glucose deficiency, remain constant irrespective of treatment conditions.

The foregoing results allowed the highlight of a preventive effect of a diet based on a composition with low polyamines content on thermal and mechanical nociceptive disorders induced by the acute administration of oxaliplatin. In addition, a diet based on a composition with low polyamines content can prevent the onset of neuropathic disorders after the administration of oxaliplatin. The introduction of a said diet 15 days after the onset of the neuropathy also allows thermal hypersensitivity to be reversed. From a mechanistic viewpoint and without being bound by any theory, the preventive effect of the diet on acute neurotoxicity induced by oxaliplatin may be due to modulation of glutamate tissue concentrations in the dorsal horn of the spinal cord.

### Example 8

### Evaluation of Spermidine, Spermine and Putrescine Erythrocyte Levels in Rats Having Received a Diet Based on a Composition with Low Polyamines Content Combined with Oxaliplatin Treatment (FIG. 7)

Three days after the injection of oxaliplatin (6 mg/kg, ip) or of the vehicle, the rats were sacrificed by decapitation. The arterial blood was then collected in tubes containing 2 ml of 0.129 M sodium citrate buffer and centrifuged at 2500 g for 10 minutes at 4° C. After removal of the plasma and the leukocyte/platelet fraction, the erythrocyte sediment was washed 3 times with 4 volumes of saline (0.9% NaCl). The proteins were then removed by adding 2 mL of cold 10% perchloric acid solution per 1 ml of erythrocytes. After an incubation time of 1 h at 4° C. and centrifuging at 3000 for 10 minutes, the perchloric supernatant was taken and analysed under HPLC using the method described by Lughezzani et at (2010).

The results in FIG. 7 are expressed in nmol/8×10⁹ erythrocytes (mean+MSD, 8 animals per group).

The results show that the concentrations of spermidine found in the red blood cells are significantly reduced in the rats treated with oxaliplatin and receiving a PDD diet.

These results appear to show synergic activity on spermidine depletion through the combination of a diet based on a composition with low polyamines content and the administration of a platinum salt such as oxaliplatin. When combined with a diet low in polyamines content, oxaliplatin appears to contribute towards depletion by acting as an inhibitor of the endogenous production of polyamines (even an activator of their catabolism) with action targeted on spermidine (Varma et al., 2007).

Therefore, the preventive effect of a diet based on a composition with low polyamines content according to the invention on acute neuropathy induced by oxaliplatin is appears to be partly caused by the oxaliplatin via its action on the production of endogenous spermidine.

### Example 9

### Measurement of the Expression of the NR2B Subunit and of Phosphorylation of the NR2B Subunit (Tyrosine 1472, Tyrosine 1336 and Serine 1303) in the Model of Acute Neuropathy Induced by Oxaliplatin

### Materials and Methods

Tissue Samples of the Dorsal Corn of the Spinal Cord

The rats given a diet containing polyamines were sacrificed by decapitation at D3 after injection of oxaliplatin (6 mg/kg, ip) and the spinal cord of the rats was quickly sampled. The dorsal corn of the spinal cord (L4-L6) was isolated on an ice block and immediately frozen in liquid nitrogen. The samples were then stored at −80° C. until analysis.

Western Blot

The tissue samples were subjected to cell lysis at 4° C. performed with 300 μL of stop buffer (50 mM Hepes, 150 mM NaCl, 10 mM EDTA, 10 mM Na₂P₂O₇, 10 mM vanadate, 2 mM Na₃VO₄, 100 mM NaF, 1% Triton, 0.5 mM PMSF, 100 UI/mL Iniprol and 20 μM Leupeptine, pH 7.5). After sonication (1 to 2 minutes at 4° C.), the sample was centrifuged at 14000 rpm at 4° C. and the supernatant collected. This centrifuging step was repeated once. The supernatant contained the cytoplasmic and membrane proteins assayed by colorimetry (BC Assay UP40840A®, Interchim). After a denaturing step at 100° C. for 5 minutes in uptake buffer (100 mM Tris, 12% SDS, 40% glycerol and 20% bromophenol/3-mercaptoethanol, pH 7.6), the proteins were separated by electrophoresis on polyacrylamide gel. The proteins were then transferred onto a nitrocellulose membrane (Millipore) for 2 hours in a transfer buffer (25 mM Tris, 190 mM glycine, 20% methanol, pH 8.3). The membranes were hybridised with the corresponding antibodies overnight at 4° C. The following antibodies were used: Total NR2B (1:500, cat#06-600, Upstate Biotechnology, Millipore, Saint-Quentin-en-Yvelines, France), phospho-Tyr1472 (1:500, Millipore, AB5403), phospho-Tyr1336 (1:500, Millipore, AB9690) and phospho-Ser1303 (1:500, Millipore, cat#07-398). The following day the membranes were hybridised with the secondary antibody coupled to the peroxidase (1:50000, Goat anti-rabbit, Pierce) diluted in blocking buffer for 1 hour under agitation. Three 10-minute washings were then carried out. Detection was made using chemiluminescence (Immun-Star™ WesternC™ Kit, Bio-rad®)). The Western-Blots were scanned with an image analyser ChemiDoc™ XRS System, Bio-rad®) and the density of each band was quantified using computer software (Image Lab™ software, Bio-rad®)). The signal of each band was normalised with the signal of the corresponding beta-actin obtained on the same membrane (1:5000, A5441, Sigma-Aldrich). The results are expressed as a percentage of the control group (non-treated group receiving a diet containing polyamines, PCD CT).

No variation in the expression of the NR2B subunit was observed between the animals treated with oxaliplatin and the control animals not treated with oxaliplatin.

No variation in phosphorylation of the NR2B subunit on the tyrosine sites 1472 and 1336 and on the serine site 1303 was observed between the animals treated with oxaliplatin and the control animals not treated with oxaliplatin.

In the light of these results, the NR2B subunit of the NMDA receptors in the spine does not appear to be involved in the physiopathology of acute neuropathy induced by oxaliplatin. The physiopathology of acute neuropathy induced by oxaliplatin is different from the physiopathology of inflammatory pain induced by an injection of carrageenan (Rivat et al., 2008). Rivat and colleagues (2008) show that the inflammation caused by an injection of carrageenan induces phosphorylation of the NR2B subunit and that a diet that is polyamine-deficient allows a reduction in the phosphorylation of this subunit.

### CITED BIBLIOGRAPHICAL REFERENCES

Andre T et al, N Engl J Med 2004; 350(23):2343-2351.
Angenstein F et al, Cereb Cortex 2008; 18(4):890-897.
Argyriou A A et al, Cancer Treat Rev 2008; 34(4):368-377.
Attal N et al, Pain 2009; 144(3):245-252.
Beckonert O et al, Nat Protoc 2007; 2(11):2692-2703.
Bardocz et al., 1995, Br J Nutr, 73(6):819-828
Burns M.R., 2009, J Med Chem, 52, 1983-1993
Chollet P et al, Ann Oncol 1996; 7(10):1065-1070.
Cipolla B G et al, Congres ESPEN, Nice, 2010,
de Gramont A et al, J Clin Oncol 2000; 18(16):2938-2947.
Fan W M T, Prog Nucl Magn Reson Spectrosc 1996; 28:161-219.
Germann N et al, Ann Oncol 1999; 10(3):351-354.
Igarashi et al., 2010, Plant Physiology and Biochemistry, 48, 506-512
Kiernan M C et al. EurJ Cancer 2007; 43(18):2631-2633.
Latremoliere A et al, J Pain 2009; 10(9):895-926.
Leveque et al., 2000, Anticancer Res, 20(1A), 97-101)
Louvet C et al, J Clin Oncol 2002; 20(23):4543-4548.
Lughezzani G et al. EurJ Cancer 2010; 46(10):1927-1935.
Monnet I et al, J Cancer 1998; 34(7):1124-1127.
Oldenburg J et al, Acta Oncol 2009; 48(6):804-806.
Pasetto L M et al, Crit Rev Oncol/Hematol 2006; 59(2):159-168.
Quenemer et al., 1995, Ann Gastroenterol Hepatol (Paris), 31(3), 181-188; discussion 188-189
Rivat C et al, Pain 2008; 137(1):125-137.
Saif M W et al, Ther Clin Risk Manag 2005; 1(4):249-258.
Soulet D et al, The Journal of Cell Biology 2003 vol. 162; no 2 July 21: 257-268
Thomas et al Cell Mol Life Sci 2001 58: 244-258)
Treede R D et al, Neurology. 2008 Apr. 29; 70(18):1630-5,
Varma R et al. Cancer Chemother Pharmacol 2007; 59(6):711-723.
Woolf C J et al, Pain 1991; 44(3):293-299.
Yen Y et al, Am J Clin Oncol-Cancer Clin Trials 2008; 31(4):317-322.
Zwingmann C et al, Neurochem Int 2005; 47(1-2):19-30.

1.-16. (canceled)

17. A food composition with low polyamines content for use in the treatment, prevention, or both treatment and prevention of neuropathic pain induced by a platinum salt, the food composition comprising a nutrient mixture comprising less than 1600 picomoles/gram of polyamines relative to the weight of the food composition.

18. The food composition according to claim 17, wherein the platinum salt is oxaliplatin.

19. The food composition according to claim 17, wherein the nutrient mixture comprises less than 400 picomoles/gram of polyamines relative to the weight of the food composition.

20. The food composition according to claim 17, wherein the nutrient mixture comprises less than 200 picomoles/gram of polyamines relative to the weight of the food composition.

21. The food composition according to claim 17, wherein the nutrient mixture comprises less than 400 picomoles/gram of spermidine relative to the weight of the food composition.

22. The food composition according to claim 17, wherein the nutrient mixture comprises less than 100 picomoles/gram of spermidine relative to the weight of the food composition.

23. The food composition according to claim 17, wherein the nutrient mixture is void of spermidine.

24. The food composition according to claim 17, wherein the nutrient mixture comprises less than 400 picomoles/gram of putrescine, less than 400 picomoles/gram of spermidine, less than 400 picomoles/gram of spermine, and less than 400 picomoles/gram of cadaverine relative to the weight of the food composition.

25. The food composition according to claim 17, wherein the nutrient mixture comprises less than 100 picomoles/gram of putrescine, less than 100 picomoles/gram of spermidine, less than 100 picomoles/gram of spermine, and less than 100 picomoles/gram of cadaverine relative to the weight of the food composition.

26. The food composition according to claim 17, wherein the nutrient mixture comprises at least one natural substance, at least one synthetic composition, or a combination thereof.

27. The food composition according to claim 26, wherein the natural substance is a food for human or veterinary use.

28. The food composition according to claim 26, wherein the synthetic composition comprises less than 1600 picomoles/gram of polyamines relative to the weight of food composition.

29. A method for inducing in a patient or an animal a reduction in excitatory glutamatergic transmission in the dorsal horn of the spinal cord comprising consuming the food composition according to claim 17.

30. A method for inducing in a patient or an animal a reduction in the tissue concentrations of glutamate, its glutamine storage form in the dorsal horn of the spinal cord, or a combination thereof comprising consuming the food composition according to claim 17.

31. A platinum salt for use in the treatment of cancer in patients or animals, wherein the patient or animal is receiving, has received, or both, a food diet low in polyamines content.

32. The platinum salt according to claim 31, wherein the food diet is low in spermidine content.

33. The platinum salt according to claim 31, wherein the platinum salt is oxaliplatin.

34. A method for anti-cancer treatment comprising administering a platinum salt to a patient or animal, wherein the patient or animal is receiving, has received, or both, a food diet comprising low polyamines content.

35. The method according to claim 34, wherein the food diet comprises low spermidine content.

36. The method according to claim 34, wherein the platinum salt is oxaliplatin.
